(19) United States
(12) Patent Application Publication
(10) Pub. No.: US 2004/0259149 A1
(43) Pub. Date: Dec. 23, 2004

(54) Sensor

(76) Inventor: Dan Veniamin Nicolau, Hawthorn (AU)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/343,728

(22) PCT Filed: Aug. 2, 2001
(86) PCT No.: PCT/AU01/00941

(30) Foreign Application Priority Data
Aug. 2, 2000 (AU) PQ 9146

(51) Int. Cl. G01N 33/53; C12M 1/34

(52) U.S. Cl. 435/7.1; 435/287.2

(57) ABSTRACT

A sensor for detection of analyte within a sample comprising a panel microfabricated to form a track or tracks having one component of a protein motor pair immobilised to a base thereof; a probe that specifically binds to the analyte is attached to a mobile component of the protein motor pair located in the track or tracks and means is provided to detect movement and/or velocity of the mobile component; wherein presence of analyte is indicated by detecting a reduction of mobile component velocity or the cessation of mobile component movement in the presence of hydrolysable nucleotide.

### FIELD OF THE INVENTION

The present invention relates to a sensor and particularly, but not exclusively, to a sensor for detecting molecular level concentrations of chemicals and/or biological agents. The invention also relates to methods of detecting and determining the presence and/or concentration of an analyte within a sample.

### BACKGROUND OF THE INVENTION

There are numerous chemical compounds and pathogens in existence that may be toxic or pathogenic to humans, plants or animals at extremely low concentrations. For example the organophosphate compounds known as sarin, soman and tabun used as nerve gasses during World War I have an average lethal dose in humans of about 0.01 mg/kg (the Merck Index, 12th Edition, 1996), exposure to hydrogen cyanide at a concentration of 300ppm for a period of just a few minutes or at a concentration of 1500ppm for between half an hour to an hour is generally lethal in humans, and the chemical weapon known as mustard gas exhibits a lethal dose in mice of 3.3 mg/kg. These are just a few simple examples of compounds that are highly toxic at extremely low concentrations. Other agents including heavy metals such as mercury, cadmium, lead and zinc, arsenic containing compounds or organic compounds such as benzene, xylene or dioxane are also considerably toxic and can have a deleterious effect on plant and animal life at relatively low concentrations, such as for example in the range of several hundred to several thousand parts per million (ppm). Biological agents such as bacteria, fungi, viruses, retroviruses and the like may also present significant toxicity or pathogenicity to animals (including humans) and plants, once again at relatively low levels.

Up to date it has been difficult to detect the low level presence of many potentially toxic or pathogenic agents or even the low level presence of agents that may present a potential contamination to a product or an industrial or manufacturing process. It is therefore desirous to develop means of detecting and/or determining the concentration of agents within samples that are present at molecular level concentrations, that is concentrations generally in the range of 1×10⁻² to 5×10⁻³ppm.

The present inventors have devised a sensor useful for determining the presence and/or concentration of an analyte within a sample. Other aspects of the present invention will become apparent from the following detailed description thereof.

### SUMMARY OF THE INVENTION

According to one embodiment of the present invention there is provided a sensor for detection of analyte within a sample comprising a panel microfabricated to form a track or tracks having one component of a protein motor pair immobilised to a base thereof; a probe that specifically binds to the analyte is attached to a mobile component of the protein motor pair located in the track or tracks and means is provided to detect movement and/or velocity of the mobile component; wherein presence of analyte is indicated by detecting a reduction of mobile component velocity or the cessation of mobile component movement, in the presence of hydrolysable nucleotide.

Preferably the panel is microfabricated to form a plurality of tracks and the means to detect velocity of the mobile component from the plurality of tracks are connected. In a preferred embodiment the measurement of reduction of mobile component velocity from the plurality of tracks allows determination of analyte concentration within the sample. In another aspect detection of instances of cessation of mobile component movement from the plurality of tracks allows determination of analyte concentration within the sample.

According to one embodiment of the invention the track or tracks is/are formed in a circuit configuration to ensure substantially unidirectional movement of the mobile component at a point or points where movement and/or velocity is detected. Preferably the circuit configuration is as shown in FIG. 3. In a preferred embodiment the movement and/or velocity detection is at one or more escape routes from the circuit configuration, provided at substantially unidirectional movement points within the circuit configuration. Preferably the escape routes are closable.

According to another embodiment of the invention the movement and/or velocity detection means comprises a magnetic bead attached to the mobile component and a loop or loops of electrically conducting material located at a point on the track or tracks where movement and/or velocity is to be detected; the loop or loops are so configured and positioned that an electrical signal is induced therein upon movement by the bead bearing mobile component at the point where movement and/or velocity is to be detected; wherein an induced signal indicates movement and/or the induced signal is a function of mobile component velocity. In one embodiment the loop or loops is/are positioned about and generally perpendicular to the track. In another embodiment the loop or loops is/are positioned adjacent and generally parallel to the track.

In a further embodiment of the invention the movement and/or velocity detection means comprises a magnetic bead attached to the mobile component and electrically conducting material located on opposite sides of the track at a point on the track where movement and/or velocity is to be detected; the conductors are configured so that when an electrical signal is applied to one conductor it results in a signal being induced at the other such that there are changes in the induced signal resulting from movement by the bead bearing mobile component at the point where movement and/or velocity is to be detected; wherein the changes in induced signal indicate movement and/or are a function of mobile component velocity.

In another embodiment of the invention the movement and/or velocity detection means comprises a fluorescent label attached to the mobile component, the movement and/or velocity of which can be detected by fluorescence imaging analysis.

In one embodiment of the invention the protein motor pair is actin/myosin. Preferably the mobile component is actin.

In another embodiment of the invention the protein motor pair is tubulin/kinesin. Preferably the mobile component is tubulin.

According to a further embodiment of the invention the protein motor pair is tubulin/dynein. Preferably the mobile component is tubulin.

In the case of tubulin containing protein motor pairs the tubulin may be in the form of a microtubule.

According to a further embodiment of the invention the panel comprises glass, silicone, ceramic or plastics material. Preferably it is glass. Preferably the glass is liquid primed.

In a further embodiment of the invention the glass, silicone, ceramic or plastics material is coated on a surface thereof with polymer.

In a still further embodiment of the invention the polymer is or includes a photoresist polymer. Preferably the photoresist polymer is selected from a diazo-naphtho-quinone (DNQ)/novalak polymer, a DNQ/novalak/imidazole polymer and a tertiary-butyl-methacrylate/methyl-methacrylate polymer.

In a preferred embodiment of the invention the analyte is a toxin or pathogen.

In another embodiment of the invention the probe is an antibody for the analyte. Preferably the antibody is a monoclonal antibody.

In another aspect of the invention binding of analyte to probe inhibits interaction between mobile and immobilised component to thereby prevent movement of mobile component relative to immobilised component.

In a further embodiment of the invention the hydrolysable nucleotide is adenosine triphosphate (ATP).

In a still further embodiment of the present invention there is provided a method of determining the presence and/or concentration of an analyte within a sample which comprises exposing the sample to a sensor comprising a panel microfabricated to form a track or tracks having one component of a protein motor pair immobilised to a base thereof; a probe that specifically binds to the analyte is attached to a mobile component of the protein motor pair located in the track or tracks and means is provided to detect movement and/or velocity of the mobile component; wherein presence of analyte. is indicated and/or concentration of analyte may be determined by detecting a reduction of mobile component velocity or cessation of mobile component movement, in the presence of hydrolysable nucleotides.

### BRIEF DESCRIPTION OF THE FIGURES

The invention will be further described with reference to the accompanying Figures in which:

FIG. 1 shows a schematic representation of a mobile component with bound probe located within a track and a mobile component with bound probe and analyte attached also located within a track. The velocity of the mobile component having analyte attached is reduced relative to that of the mobile component with no analyte attached; and

FIG. 2 shows schematic representations of two velocity detection means encompassed by the invention. In the upper part of the figure there is shown a loop of electrically conducting material located about and generally perpendicular to the tracks and in the lower part of the figure there is shown a loop of electrically conducting material located beneath and generally parallel to the tracks; and

FIG. 3 shows a schematic representation of a circuit configuration of track that may be utilised to ensure substantially unidirectional mobile component movement.

FIG. 4(a) shows a diagrammatical representation of movement of mobile component/probe relative to the immobilised component and (b) diagrammatically shows an example of the invention wherein the probe is bound to the immobilised component in a manner such that when analyte is bound-to the probe the interaction between the mobile and immobilised components will be interrupted.

### DETAILED DESCRIPTION OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference to any prior art in this specification is not, and should not be taken as, an acknowledgment or any form of suggestion that that prior art forms part of the common general knowledge in Australia.

Throughout this specification the agent that is to be detected or quantifiably detected within a sample will be referred to as an “analyte” and in this context the term “sample” is intended to embrace samples of gas or liquid, solutions, extracts from soil, plant or animal matter or extracts of industrial materials or wastes such as for example mineral slags or slurries, industrial chemical materials, foods, drinks, pharmaceutical, veterinary or agrochemical products or components used in the manufacture thereof. In particular, the term “sample” is intended to encompass air and water obtained from lakes, streams, rivers, estuaries, the sea or the like.

By utilising the sensor according to the present invention it is possible to detect the presence and/or concentration of a vast array of analytes. The main limitations are that there must be a probe that specifically binds to the analyte that can be attached to a mobile component of the protein motor pair incorporated within the sensor and that when this probe is attached to the mobile component, the analyte should bind to it with the binding affinity strongly favouring the binding of probe and analyte over the binding of probe with any other species. The presence and/or concentration of analytes including toxins such as heavy metals, pesticides, herbicides, pharmaceutical agents, veterinary agents, industrial or manufacturing biproducts, solvents, chemical warfare agents, metabolites or unwanted impurities, degradation products or the like or biological agents such as bacteria, fungi, viruses, retroviruses, parasites or other pathogens can be determined using the sensor of the present invention.

The basic structure of the sensor according to the invention is provided by a microfabricated panel. The panel may take a variety of different forms. For example it may be etched such as by wet etching or reactive ion etching, it may be embossed or stamped using a variety of microlithography techniques onto a glass, silicone, ceramic or plastics material. Generally the material will be in the form of a sheet or plate. It is also possible using plastics material for the microfabricated panel to be formed by microfabricating injection moulding techniques. Similarly, it is possible for the panel to be produced from glass, silicone, ceramic or plastics material and to then have applied to a surface thereof a microfabrication polymer or polymers which may for example be applied in layers. In a preferred aspect of the invention the polymer or polymers will include a photoresist polymer that will allow for the formation of a microstructure on the surface of the panel, involving the use of radiation. Details of known techniques for preparing microfabricated panels and surfaces are provided in Handbook of Microlithography, Micromachining & Microfabrication, editor P. Ray-Choudhury, Vol. 1 & 2, SPIE Press, 1997, the disclosure of which is included herein in its entirety by way of reference. Some particular examples of photoresist polymers that may be utilised in this fashion include diazo-naphtho-quinone (DNQ)/novalak polymers, DNQ/novalak/imidazole polymer and tertiary-butyl-methacrylate/methyl-methacrylate polymer.

An important aspect of the present invention is that a track or tracks are formed within the panel. The intention of these tracks is to contain and control the direction of motion of a mobile component of a protein motor pair. The tracks microfabricated within a surface of the panel will therefore preferably be between about 20 nm and about 600 nm in width and between about 40 nm and about 800 nm in depth. Particularly preferably the tracks are between about 40 nm and 200 nm in width and between about 80 and about 400 nm in depth Preferably the track or tracks will have a substantially straight side edge and base profile.

Protein motor pairs are pairs of proteins or polypeptides able to obtain energy via nucleotide hydrolysis and to use this energy to perform mechanical work. In biological systems protein motor pairs are responsible for muscular activity, the movement of flagella and cilia and intracellular movements such as exocytosis and mitosis. Some details of known motor proteins are provided in Molecular Biology and Biotechnology, a Comprehensive Desk Reference, edited by Robert A Myers, pp. 564-569 (Motor Proteins by Elluru, R. G., Cyr, J. L. and Brady, S. T.) the disclosure of which is included herein in its entirety by way of reference. Three families of well-known protein motor pairs are the kinesin, dynein and myosin protein families. Myosin interacts with microfillaments composed substantially of actin and kinesin and dynein interact with microtubules generally comprised of heterodiomers of α- and β-tubulins and a variable set of associated proteins. The known protein motor pairs are capable of movement between components of the pair by hydrolysis of nucleotides. The known protein motor pairs hydrolyse adenosine triphosphate (ATP) and are therefore known as ATPases. The present invention is by no means limited to the use of known protein motor pairs but may encompass the use of protein motor pairs as yet undiscovered. The present invention also is not limited to the use of ATP as an energy source but may involve the use of other hydrolysable nucleotides. Components of protein motor pairs can be produced, for example by recombinant DNA technology in an appropriate host cell or may be extracted from a suitable animal, plant or microorganism.

The protein motor pairs that may be incorporated within the sensor of the invention will have one component immobilised to the base of the track or tracks and will have a second component able to migrate along and within the track adjacent to the immobilised component, which will be referred to as the mobile component. It is possible for either of the components of each protein motor pair to be immobilised to the base of the tracks, although it is preferred for the mobile component to constitute the filamentous component that is, actin in the case of the actin/myosin protein motor pair and tubulin in the case of both the tubulin/kinesin and tubulin/dynein protein motor pairs. In relation to protein motor pairs involving tubulin, and to assist in directionally constraining movement of the mobile component of the pair, tubulin may be utilised in the form of microtubules, an agglomeration of tubulin dimers and other protein having a tubular structure of approximately 24 mm in diameter.

The base of the track or tracks can be functionalised if necessary and can have a component of the protein motor pair immobilised to it by the use of well known chemistry. Examples of approaches that may be taken to immobilise a component of a protein motor pair to the base of a track are outlined in Nicolau, D. V. et al “Positive and Negative Tone Protein Patterning Using Conventional Deep-UV/E-beam Resists”, Langmuir, 15, 3845-3851, 1999; Nicolau, D. V. “Micron-size Protein Patterning on Diazo-naphtho-quinone/novalak Polymeric Films” Langmuir, 14, 1927-1936, 1998; Nicolau, D. V. Protein Profiled Features Patterned Via Confocal Microscopy”, Biosensors & Bioelectronics, 15 (2000) 85-92 and Nicolau, D. V. et al “Protein Patterning via Radiation-assisted Surface Functionalisation of Conventional Microlithographic Materials”, Colloids and Surfaces A: Physicochemical and Engineering Aspects 155 (1999) 51-62, the disclosures of which are included herein in their entirety by way of reference. Protein may also be immobilised onto the base of the tracks by way of adsorption, such as for example explained in “Protein Adsorption”, Edited by Andrade, J. D., Plenium Press, New York, 1985 the disclosure of which is also included herein in its entirety by way of reference. Other means of immobilising proteins onto solid state surfaces (eg. glass, silicone, ceramics which may include Si and/or SiO₂ functionalities) are explained in Pirrung, M. C., Yang C. H., A General Method for the Spatially-defined Immobilisation of Biomolecules on Glass Surfaces Using Caged Biotin, Biocongate Chem. 7, 317-221 (1996) and F. S. Ligler, Rowe, C. A., Balderson, G. A., Feldstein, M. J., Golden J. P., Fluorescence Array Biosensors Part 2, Biochemistry and Application Micro Total Analysis Systems 1998 Edited by Harris, D. J. & Van den Berg, A., Kluweiz Academic Publ. 217-220, the disclosures of which are also included herein in their entirety by way of reference.

A probe that specifically binds to the analyte is attached to the mobile component of the protein motor pair. The probe will be selected specifically in each case depending upon the analyte which is intended to be detected so that the probe specifically binds the analyte and so there is sufficient affinity between the analyte and the probe favouring this binding interaction such non-specific binding between the probe and other entities is unlikely to adversely affect detection and/or concentration determination of the analyte. The probe may take a variety of different forms such as for example inorganic or organic compounds that act as ligands for other chemical species such as for example the use of porphyrin compounds to bind heavy metals. In particular, the use of antibodies as probes is favoured if it is possible for antibodies to be generated against the analyte of interest. Antibodies utilised within the invention may be polyclonal or preferably monoclonal antibodies. Production of antibodies against an analyte of interest can be achieved by well recognised methods such as for example those discussed in Mayer R. J. & Walker J. H., immunochemical Methods in Cell and Molecular Biology, Academic Press Limited, 1987 and as also recited in Ausubel et al (1987), In: Current Protocols in Molecular Biology, Wiley Int. Science, the disclosures of which are included herein in their entirety by way of reference. The probes according to the invention can be linked to the mobile component by routine chemical methods. For example, via covalent linkage which may utilise carboxy, amino or hydroxyl moieties, via hydrophobic non-specific attachment, via biotin/avidin functionalisation, antibody/antigen interaction or even via attachment to bead or other linker as mentioned below.

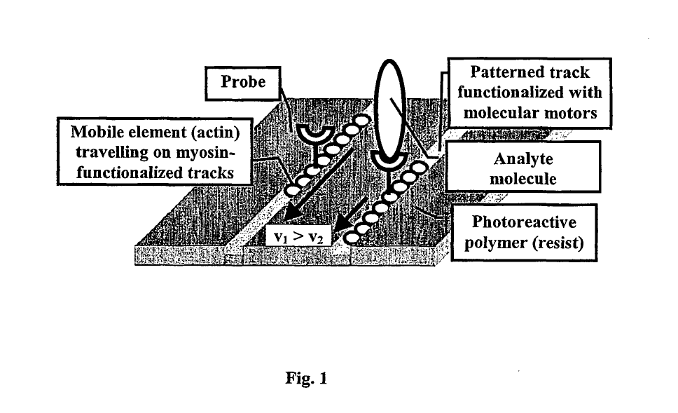

The sensor according to the invention includes means to detect movement and/or velocity of the mobile component. One rationale for this is that velocity of the mobile component when the probe is bound to an analyte will be reduced relative to the situation where no analyte is bound to the probe. Therefore, by determining if velocity of the mobile component is slowed relative to the velocity when no analyte is present it is possible to determine that analyte is present in a particular sample. This aspect of the invention is schematically represented in FIG. 1. Similarly, and by having a plurality of tracks within which protein motor pairs are located and to which a probe specific for the analyte of interest is attached to the mobile component, it is possible to determine concentration of the analyte by determining the ratio of mobile components reduced in velocity relative to those that do not experience a velocity change. Similarly, binding of analyte to the probe may prevent altogether movement of the mobile component relative to the immobilised component. In this case the cessation of movement indicates that analyte is present and by determination of the proportion of tracks/protein motor pairs where movement has ceased relative to those where it has not it is possible to determine analyte concentration.

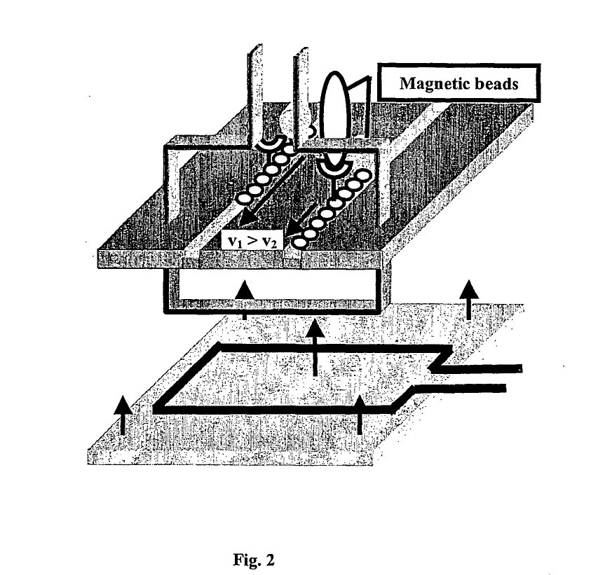

The means for detecting velocity of the mobile component may take a variety of forms. In one aspect of the invention a loop of electrically conducting material is provided about the track at a point where movement and/or velocity is to be detected, with the loop generally located perpendicular to the track. This aspect of the invention is shown diagrammatically in the top part of FIG. 2. In conjunction with this type of arrangement a magnetic bead is also attached to the mobile component such that when the mobile component migrates through the loop an electric signal will be induced within the loop and can be detected. For example the magnetic bead may be a paramagnetic bead with a diameter of 1 μm and a magnetisation for 20% Fe₂O₃ of 12.7KA/m. For example, magnetic beads may be attached by functionalisation with Gelsolin or the use of biotin/avidin functionalisation, as is well understood in the art. Functionalised probes (eg. biotinylated) may be in turn attached to the bead. The amplitude and/or the wavelength of the signal may be utilised to determine velocity of the mobile component, which by comparison to velocity of mobile component not bound to analyte will provide an indication of the presence or absence of analyte. Preferably, numerous loops of the type referred to above are in electrical communication so that what may be a relatively small electrical signal obtained from each track is amplified to a measurable level, so that concentration of the analyte can be determined. By a similar approach a loop may equally be positioned generally parallel to the track, for example beneath, above or to a side of the track. A diagrammatic representation of this aspect of the invention is shown in the lower part of FIG. 2. In the case of a loop positioned about and generally perpendicular to the track this may be formed by microfabrication microcircuitry whereas in the case of a loop located generally parallel to the track it may be possible for an imprinted wire frame to be incorporated into the microfabricated panel, for example beneath the or each track.

In another aspect of the invention electrical conducting materials are located on opposite sides of the track and an electrical signal is applied to one of the conductors resulting in a signal being induced at the other. When a mobile component carrying a magnetic bead migrates past these conductors there will be changes in the induced signal that can be detected. These changes in the induced signal are a indicative of mobile component movement and function of the mobile component velocity, from which the mobile component velocity can be determined. In this case the electrically conducting material located on opposite sides of the track may constitute, for example a miniature plate, loop or coil of electrically conducting material.

The means to determine movement and/or velocity of the mobile component could also involve the use of a fluorescent label bound to the mobile phase, the movement and/or velocity of which may be determined using fluorescence imaging analysis. Examples of suitable fluorescent labels include tetramethyl rhodamine conjugated to phalloidin (see Kron & Spudich, Proc. Natl. Acad. Sci. USA, 83 (1986), 6272-6276) as well as ethidium bromide, FITC (fluorescein-isothiocyanate) and others such as mentioned in Lakowicz. R, 1993, Principles of Fluorescence Spectroscopy, Plenum Press, NY, USA or as commercially available from Molecular Probes, Inc (see www.probes.com). Fluorescent labels such as these can be conjugated to the mobile component by standard chemistry techniques, such as already mentioned in relation to probes.

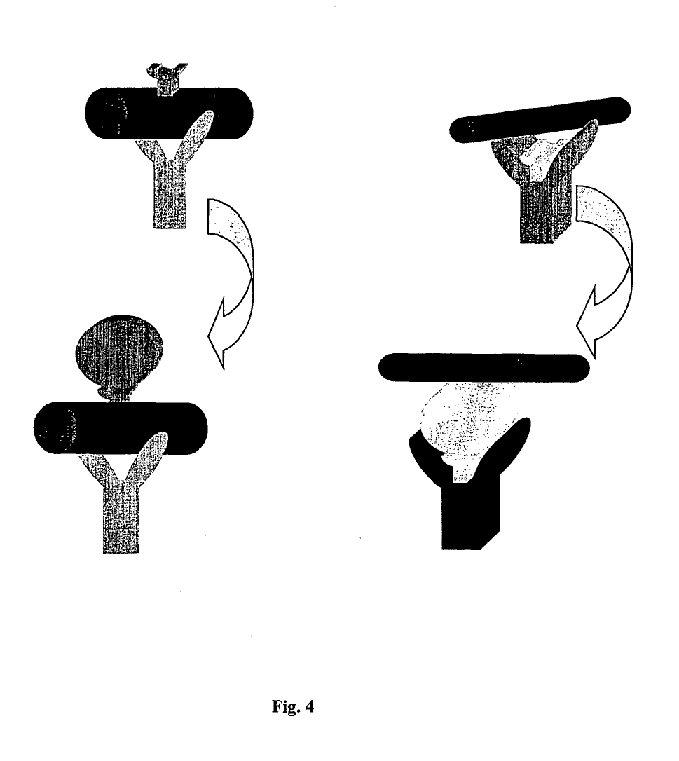

In another aspect of the invention the probe may be bound to either the mobile or immobilised component in a position such that when analyte is in turn bound to the probe the interaction between the mobile and immobilised components is interrupted. In this way movement between the mobile and immobilised components will be prevented when analyte is bound to the probe. For example, this approach is shown in FIG. 4. In part (a) of FIG. 4 where the immobilised component 1 has the mobile component 2 located adjacent and in a position to move relative to it. The probe 3 is shown both unbound and bound to analyte 4, the binding of which will either slow or prevent movement of the mobile component relative to the immobilised component. In part (b) the probe 3 is bound within the section of the immobilised component 1 which interacts with the mobile component 2, but when no analyte 4 is present the mobile component 2 is free to move relative to the immobilised component 1. However, in the situation where analyte 4 is bound to the probe 3 the interaction between mobile component 2 and immobilised component 1 is interrupted such that relative movement is prevented. In part (b) a fluorescent label or bead 5 used to detect movement of the mobile component 2 is bound to the mobile component 2. In a preferred example of this embodiment of the invention the probe may be bound within the forked arms of the myosin molecule thus preventing the action/myosin interaction in the case when analyte is bound to the probe.

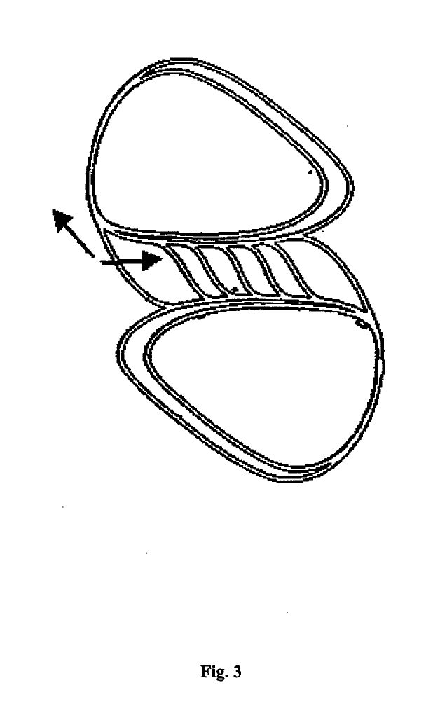

In a preferred embodiment of the invention each individual track is configured into a, circuit configuration that will ensure that at given points within the circuit mobile components located therein are travelling substantially in a single direction. An example of a circuit configuration designed to achieve this end is shown in FIG. 3 where, as can be seen, the configuration will favour generally clockwise movement of mobile components within the upper circle and generally clockwise movement of mobile components within the lower circle. Within such a configuration it is also possible to introduce “escape routes” that can be opened or closed. The velocity detection means may conveniently be positioned on or adjacent one of these escape routes. Electrical connection in series of these velocity detection means will produce a unidirectional signal. For example, escape routes could conveniently be positioned at the left hand side of the upper circle heading upwards or the right hand side of the lower circle heading downwards in relation to the circuit configuration shown in FIG. 3.

Other methods may also be used in combination with the use of circuit configured tracks for movement control. For example movement may be minimalised by reducing the operation temperature of the system to below room temperature and preferably to between 0° C. and 10° C., particularly preferably between 0° C. and 5° C. It is also possible to minimise the movement of the mobile component by reducing concentrations of hydrolysable nucleotide. Generally hydrolysable nucleotide concentrations (for example ATP) will be in the order of those found in animal cells.

In another aspect of the invention the presence of electrically conducting material on opposite sides of the track may be used as direction controllers by their operation intermittently and for short durations between an electric generator and an electric motor mode. Once the mobile components are redirected via control of the movement of the attached magnetic bead they will then follow the newly assigned direction until redirected again.

In use the sensor according to the invention may be adapted to be directly exposed to the sample of interest such as for example by immersion in a liquid sample or direct exposure to a gas where particles within the gas will be free to partition across a solution in which the sensor may be bathed. The sensor may for example be adapted to give a colour display or electronic yes/no or numeric output, obviously with the appropriate circuitry and power supply. The sensor may also be equipped with minute transmitter and antenna and optionally microprocessor to transmit data on analyte presence/concentration to another location where it may be read and/or collated with other data. The sensor may be adapted to be worn by an individual and attached, for example to an individual's arm or leg by a strap or adhesive or could be incorporated into an individual's clothing, or uniform, particularly protective clothing.

In a preferred embodiment of the invention the sensor may take the form of an implantable biochip that may be implanted within a living system or it could be permanently inserted within an industrial or manufacturing process line or within quality maintaining equipment. In such cases it may be desirable for the sensor to be separated from its immediate environment for example by a semi permeable membrane.

The sensor may also take the form of a microfluid chip where the sensor is a component of an assembly where fluid (liquid or gas) is injected into the sensor through a microfabricated “port”.

It is to be understood that the present invention has been described by way of example only and that modifications and/or alterations thereto that would be readily apparent to a person skilled in the art based upon the disclosure herein are also considered to fall within the scope and spirit of the invention.

### EXAMPLES

The present invention will now be further described with reference to the following examples:

### Example 1

### Preparation of the Protein-Selective Polymer Surfaces

The radiation sensitive material used is a copolymer of tert-butyl methacrylate (tBuMA) with methyl methacrylate (MMA). The copolymer is sensitive to the e-beam radiation and deep-UV light. 4 inch silicon wafers were (i) liquid-primed with hexamethyldisilazane (purchased from Aldrich Colo.); (ii) spin coated with a 5% polymer solution at a rotation speed of 3000 rpm to form uniform films approximately 0.6 μm thick; (iii) soft-baked at 85° C. in a convection oven for 3 hrs.; (iv) pattern exposed with an e-beam exposure machine (ZBA 21, Jenoptik, Germany) using a test pattern with exposure energy around 5 μC/cm². The wafers were cut in 1 cm² squares to accommodate the further processes of the selective attachment of the proteins. A solution of heavy meromyosin was deposited on the surface of the patterned exposed resist.

### Example 2

### Preparation of Molecular Motor Pairs

Heavy meromyosin (HMM) and actin, were extracted from the back and leg muscle of a rabbit and purified by the methods previously reported (S. S. Margossian, S. Lowey. Methods Enzymol. 85, pp. 55-71, 1982). The actin filament was labelled with tetra-methyl-rhodamine-phalloidin for fluorescence observation. The assay buffer solution used to bathe the molecular motor pair consisted of 40 mM KC1, 3mM MgCl₂, 2 mM ethyleneglycol-bis-(2-aminoethylether)-N,N,N′,N′-tetraacetic acid (EGTA), 10 mM dithiothreitol (DTT), and 20 mM N-2-hydroxyethylpiperazine-N′-2-ethanesulfonic acid (HEPES) (pH 7.8).

### Example 3

### Preparation of Sensor

The patterned P(tBuMA-co-MMA) resist surface was used as a scaffold for protein selective attachment. The observation cell consists of a glass coverslip on which the 1 cm² piece of silicon wafer with the patterned polymer surface on top was fixed with an adhesive, and a nitrocellulose-coated coverslip. Two parallel lines of grease were placed on the both sides of the silicon wafer as spacers for the buffer solution. A drop of a solution of HMM (0.1 mg/ml in the assay buffer) was placed onto the surface of the patterned polymer and then the cell was covered with the nitrocellulose-coated coverslip. HMM molecules were selectively adsorbed onto the polymer surface during a 5 min contact time. Unbound HMM molecules were washed from the cell by infusing the assay buffer solution from one side of the cell. Finally the assay buffer solution containing actin filaments labelled with tetramethylrhodamine-phallodin, 1 mM ATP, 5 mg/ml glucose, 50 μg/ml glucose oxidase and 10 μg/ml catalase was introduced into the cell.

### Example 4

### Data Collection and Analysis

Actin filaments moving on the surface were observed at room temperature (24- 25° C.) with an epifluorescence microscope (Olympus BX-50) and recorded with an image-intensified CCD camera system (Hamamatsu Photonics C2400-87). The recorded images were further processed and statistically analyzed using an image analysis software (Retrac, University of York, UK). The coordinates of the consecutive positions were used to compute instantaneous velocity, and thereby determine analyte concentration.

1. A sensor for detection of analyte within a sample comprising a panel microfabricated to form a track or tracks having one component of a protein motor pair immobilised to a base thereof; a probe that specifically binds to the analyte is attached to a mobile component of the protein motor pair located in the track or tracks and means is provided to detect movement and/or velocity of the mobile component; wherein presence of analyte is indicated by detecting a reduction of mobile component velocity or the cessation of mobile component movement, in the presence of hydrolysable nucleotide.

2. The sensor according to claim 1 wherein the panel is microfabricated to form a plurality of tracks and the means to detect movement and/or velocity of the mobile component from the plurality of tracks are connected.

3. The sensor according to claim 1 wherein measurement of the reduction of mobile component velocity from the plurality of tracks allows determination of analyte concentration within the sample.

4. The sensor according to claim 1 wherein detection of instances of cessation of mobile component movement from the plurality of tracks allows determination of analyte concentration within the sample.

5. The sensor according to claim 1 wherein the track or tracks is/are formed in a circuit configuration to ensure substantially unidirectional movement of the mobile component at a point or points where movement and/or velocity is detected.

6. The sensor according to claim 5 wherein the circuit configuration is as shown in FIG. 3.

7. The sensor according to claim 4 wherein movement and/or velocity detection is at one or more escape routes from the circuit configuration, provided at substantially unidirectional movement points within the circuit configuration.

8. The sensor according to claim 6 wherein the escape routes are closeable.

9. The sensor according to claim 1 wherein the movement and/or velocity detection means comprises a magnetic bead attached to the mobile component and a loop or loops of electrically conducting material located at a point on the track or tracks where movement and/or velocity is to be detected; the loop or loops are so configured and positioned that an electrical signal is induced therein upon movement by the bead bearing mobile component at the point where movement and/or velocity is to be detected; wherein an induced signal indicates movement and/or the induced signal is a function of mobile component velocity.

10. The sensor according to claim 9 wherein the loop or loops is/are positioned about and generally perpendicular to the track.

11. The sensor according to claim 9 wherein the loop or loops is/are positioned adjacent and generally parallel to the track.

12. The sensor according to claim 1 wherein the movement and/or velocity detection means comprises a magnetic bead attached to the mobile component and electrically conducting material located on opposite sides of the track at a point on the track where movement and/or velocity is to be detected; the conductors are configured so that when an electrical signal is applied to one conductor it results in a signal being induced at the other such that there are changes in the induced signal resulting from movement by the bead bearing mobile component at the point where movement and/or velocity is to be detected; wherein the changes in induced signal indicate movement and/or are a function of mobile component velocity.

13. The sensor according to claim 1 wherein the movement and/or velocity detection means comprises a fluorescent label attached to the mobile component, the movement and/or velocity of which can be detected by fluorescence imaging analysis.

14. The sensor according to claim 1 wherein the protein motor pair is actin/myosin.

15. The sensor according to claim 1 wherein the protein motor pair is tubulin/kinesin.

16. The sensor according to claim 1 wherein the protein motor pair is tubulin/dynein.

17. The sensor according to claim 14 wherein the mobile component is actin.

18. The sensor according to claim 15 wherein tubulin is in the form of a microtubule.

19. The sensor according to claim 16 wherein tubulin is in the form of a microtubule.

20. The sensor according to claim 15 wherein the mobile component is tubulin.

21. The sensor according to claim 16 wherein the mobile component is tubulin.

22. The sensor according to claim 1 wherein the panel comprises glass, silicone, ceramic or plastics material.

23. The sensor according to claim 22 wherein the panel comprises glass.

24. The sensor according to claim 22 wherein the glass is liquid primed.

25. The sensor according to claim 22 wherein the glass, silicone, ceramic or plastics material is coated on a surface thereof with polymer.

26. The sensor according to claim 25 wherein the polymer is or includes a photoresist polymer.

27. The sensor according to claim 26 wherein the photoresist polymer is selected from a diazo-naphtho-quinone (DNQ)/novalak polymer, a DNQ/novalak/imidazole polymer and a tertiary-butyl-methacrylate/methyl-methacrylate polymer.

28. The sensor according to claim 1 wherein the analyte is a toxin or pathogen.

29. The sensor according to claim 1 wherein the probe is an antibody for the analyte.

30. The sensor according to claim 29 wherein the antibody is a monoclonal antibody.

31. The sensor according to claim 1 wherein binding of analyte to probe inhibits interaction between mobile and immobilised component to thereby prevent movement of mobile component relative to immobilised component.

32. The sensor according to claim 1 wherein the hydrolysable nucleotide is adenosine triphosphate (ATP).

33. A method of determining the presence and/or concentration of an analyte within a sample which comprises exposing the sample to a sensor comprising a panel microfabricated to form a track or tracks having one component of a protein motor pair immobilised to a base thereof; a probe that specifically binds to the analyte is attached to a mobile component of the protein motor pair located in the track or tracks and means is provided to detect movement and/or velocity of the mobile component; wherein presence of analyte is indicated and/or concentration of analyte may be determined by detecting a reduction of mobile component velocity or cessation of mobile component movement, in the presence of hydrolysable nucleotides.
